(19) United States
(12) Patent Application Publication
(10) Pub. No.: US 2022/0304449 A1
(43) Pub. Date: Sep. 29, 2022

(54) FACIAL TREATMENT SYSTEMS AND METHODS

(71) Applicant: JPL Products LLC, Brookhaven, GA (US)

(72) Inventors: Hong Hai TRUONG, Brookhaven, GA (US); Paul Tri Luu TRAN, Brookhaven, GA (US)

(21) Appl. No.: 17/458,975

(22) Filed: Aug. 27, 2021

Related U.S. Application Data
(60) Provisional application No. 63/165,735, filed on Mar. 24, 2021.

(51) Int. Cl. A45D 34/04; A61K 8/9789; A61Q 19/00; A61K 8/04

(52) U.S. Cl. CPC A45D 34/041; A61K 8/9789; A61Q 19/007; A61K 8/044; A61K 2800/87; A61K 2800/5922

(57) ABSTRACT

A method for applying an emollient fluid to the skin includes cooling an emollient fluid and applying the cooled fluid to the skin. The emollient fluid may be made from a powdered plant product mixed into an aqueous medium. The fluid may be cooled to form a solid which is applied to the skin. Optionally, the solid is formed on an applicator which serves as a handle for the user. A kit which includes a mold, an applicator and a supply of emollient product is provided. The powdered plant product may be made from a powdered plant, powdered fruit or powdered extract of any one or more of pineapple, pumpkin, rice, banana, oats, cucumber, strawberry, lemon peel, watermelon, guava, mango avocado, tomato, blueberries, pomegranate, green tea (matcha), spirulina, dragon fruit, and beet.

### CROSS-REFERENCE TO RELATED APPLICATIONS

This application claims the benefit of U.S. provisional application No. 63/165,735, filed Mar. 24, 2021, the contents of which are incorporated herein by reference in their entirety.

### FIELD OF THE INVENTION

The present disclosure relates generally to skincare, and more particularly, to a skincare product and method and kit or system for applying the skincare composition to the skin.

### BACKGROUND

Superfood skincare focuses on using effective natural ingredients to improve the health and appearance of skin. Often plant-based, these ingredients are nutritionally dense and may be applied directly to the skin instead of ingested, for example, as a face mask. Ice, another skincare tool, has also been used as a therapy to promote clear skin and a healthy-looking glow. Cryotherapy and similar treatments focus on lowering the temperature of skin and/or the underlying tissue to provide benefit.

### SUMMARY

In one general embodiment this disclosure provides a method for applying an emollient fluid to the skin, comprising cooling the emollient fluid to a temperature below room temperature; and contacting the cooled emollient fluid to the skin.

The emollient fluid may comprise a volume of powdered plant product mixed into an approximately equal volume of an aqueous medium.

The method may include cooling the emollient fluid to a solid emollient; and contacting the solid emollient to the skin to thaw a portion of the solid emollient and deposit a layer of the emollient fluid on the skin.

The method optionally comprises providing an applicator having a first portion and a second portion and inserting the first portion of the applicator into the emollient fluid before cooling the emollient fluid, and cooling the emollient fluid while the second portion of the applicator extends out from the fluid, to form the solid emollient on the first portion of the applicator, and grasping the second portion of the applicator to contact the solid emollient with the skin.

In one embodiment, the method includes forming an emollient fluid by combining the powdered plant product with the aqueous medium.

In a particular embodiment the method comprises disposing the emollient fluid in the concave portion of a container unit; inserting the first portion of an applicator into the emollient fluid in the concave portion of the container unit with a second portion of the applicator extending out from the fluid; cooling the emollient fluid in the container unit to a solid emollient on the first portion of the applicator; removing the solid emollient and the applicator from the container unit; and using the second portion of the applicator to contact solid emollient with the skin.

According to one aspect, the powdered plant product comprises powdered banana fruit extract; powdered papaya fruit extract; powdered turmeric, and powdered aloe vera. For example, the powdered plant product may comprises about 80% powdered banana fruit extract, about 15% powdered papaya fruit extract, about 2.5% powdered tumeric and about 2.5% powdered aloe vera, by weight of the powdered plant product.

According to another aspect, this disclosure provides a skin treatment kit comprising a mold having a concave portion, an applicator having a first portion and a second portion; and an emollient product.

The emollient product may comprise a powdered plant product and/or an emollient fluid comprising powdered plant product mixed into an approximately equal volume of an aqueous medium such as water or rose water.

Alternatively, in any of the foregoing embodiments, the powdered plant product may comprise one or more powdered dried plant or powdered dried plant extracts selected from the group comprising pineapple, pumpkin, rice, banana, oats, cucumber, strawberry, lemon peel, watermelon, guava, mango avocado, tomato, blueberries, pomegranate, green tea (matcha), spirulina, dragon fruit, and beet.

In one embodiment of a kit, the container unit has a concave portion and a rim surrounding the concave portion, and the applicator comprises a closure flange between the first portion and the second portion, and the closure flange is configured to removably engage the rim while the first portion of the applicator is disposed in the concave portion of the container unit.

Other embodiments, features, and aspects of the disclosed invention are described in detail herein and are considered a part of the disclosed invention. Other embodiments, features, and aspects can be understood with reference to the following detailed description, accompanying drawings, and claims.

### BRIEF DESCRIPTION OF THE DRAWINGS

A more complete appreciation of the present disclosure and many of the attendant advantages thereof will be readily obtained as the same becomes better understood by reference to the following detailed description when considered in connection with the accompanying drawings, wherein:

FIG. 1 depicts a kit including a mold and an applicator according to a first embodiment.

FIG. 2 depicts a tray for holding multiple molds, according to a second embodiment.

FIG. 3 depicts two pops being removed from two molds supported in the tray of FIG. 2.

### DETAILED DESCRIPTION

Embodiments of the disclosed invention include systems, methods, and kits for treating skin, optionally facial skin, with an emollient product. In certain embodiments, the emollient product is a fluid which is cooled until at least partially frozen into a solid before being applied to the skin, sometimes by using an applicator frozen into the emollient fluid (sometimes referred to herein as a “topical solution”) to deposit solid emollient (sometimes referred to herein as “frozen topical solution”) on the applicator. Such combination of the solid on the applicator may be referred to herein as a “pop”.

Although an example pop may include an at least partially frozen topical solution in some embodiments, an actual phase change does not necessarily have to occur in or to some or all of the topical solution in other embodiments. Instead, merely lowering a temperature of the treatment below a normal temperature of the body, or a customary room temperature, may be sufficient to impart therapeutic benefits, such as a cooling sensation (e.g., through heat exchange), or a reduction in apparent inflammation (e.g., due to vasoconstriction). Moreover, lowering a temperature of a topical solution can change physical properties of the solution without freezing it. Thus, an emollient product which is a solid or paste at room temperature may be cooled for application to the skin, or the applicator, optionally in the form of a brush, may be used to apply a cooled emollient fluid to the skin. For example, a wax- or butter-based solution may solidify or harden at relatively lower temperatures, allowing it to better hold a certain shape (e.g., a hemispherical or roller-like shape) for more even and effective application to the face.

Although certain embodiments are herein described in the context of a facial treatment, certain embodiments can include selection, mixing, cooling, and application of topical solutions to other parts of the body or types of skin. As described further herein, a topical solution used in the treatment may be customized to fit a skin profile of a subject, including localized skin issues, and changing skin issues of a same subject over time.

Some embodiments of the disclosed invention will be described more fully hereinafter with reference to the accompanying drawings. The disclosed invention may, however, be embodied in many different forms and should not be construed as limited to the embodiments set forth herein.

In the following description, numerous specific details are set forth. However, it is to be understood that some embodiments of the disclosed invention may be practiced without these specific details. In other instances, well-known methods, structures, and techniques have not been shown in detail in order not to obscure an understanding of this description. References to “one embodiment,” “an embodiment,” “example embodiment,” “some embodiments,” “certain embodiments,” “various embodiments,” etc., indicate that the embodiment(s) of the disclosed invention so described may include a particular feature, structure, or characteristic, but not every embodiment necessarily includes the particular feature, structure, or characteristic. Further, repeated use of the phrase “in one embodiment” does not necessarily refer to the same embodiment, although it may.

Throughout the specification and the claims, the following terms take at least the meanings explicitly associated herein, unless the context clearly dictates otherwise. The term “or” is intended to mean an inclusive “or.” Further, the terms “a,” “an,” and “the” are intended to mean one or more unless specified otherwise or clear from the context to be directed to a singular form.

Unless otherwise specified, the use of the ordinal adjectives “first,” “second,” “third,” etc., to describe a common object, merely indicate that different instances of like objects are being referred to, and are not intended to imply that the objects so described must be in a given sequence, either temporally, spatially, in ranking, or in any other manner.

Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure.

Creating balanced, effective mixes of superfoods for topical application can be difficult for consumers looking for consistent and noticeable results. Moreover, cryotherapy and similar treatments can pose danger to participants though overexposure of skin or other sensitive tissues to extreme temperatures. Some or all of these deficiencies may be addressed by certain embodiments of the present invention. Certain embodiments include methods, systems, and kits for treating skin using combinations of superfood and cryotherapy. In some embodiments, a superfood mask can be applied to the skin in its frozen form, allowing the mask to melt onto the face as its applied. Accordingly, a subject can receive the benefits of both topical application and cryotherapy in one treatment. Moreover, some of these embodiments can be practiced by consumers to achieve therapeutic or cosmetic effects in a typical home environment.

According to an example embodiment, a method is provided for applying an emollient fluid to the skin, the method comprising cooling the emollient fluid to a temperature below room temperature; and contacting the cooled emollient fluid to the skin. The emollient fluid may comprise a volume of powdered plant product mixed into an approximately equal volume of an aqueous medium. The powdered plant product may be powdered plant or fruit or powdered extracts thereof, from pineapple, pumpkin, rice, banana, oats, cucumber, strawberry, lemon peel, watermelon, guava, mango avocado, tomato, blueberries, pomegranate, green tea (matcha), spirulina, dragon fruit, and beet or other beneficial plants.

The method may include combining powdered plant product with an aqueous medium such as water in a container unit such as a mold, to form an emollient fluid which may be a liquid or paste. The amounts of powder and aqueous medium can be selected according to the user's preference for a past or liquid, and whether the paste or liquid will solidify at the cooling temperature selected by the user. In one embodiment, a combination of a powdered plant product comprising 80% powdered banana fruit extract, 15% powdered papaya fruit extract, 2.5% powdered tumeric and 2.5% powdered aloe vera, by weight of the powdered plant product, mixed with an equal volume of water, was found to create a topical solution which formed a pop on an applicator and deposited a mask of emollient fluid on a user's face when the pop was applied to the skin as described herein. The proportions stated for components of the powder and of the topical solution need not be exact. It is expected that a satisfactory emollient fluid can be prepared in volume proportions of powdered plant product to aqueous media in a range from 25:75 or thereabout to 75:25 or thereabout, e.g. from about 30:70 or about 40:60 or about 50:50. Similarly, the proportions of ingredients in the powdered plant product are not critical.

The method may further include mixing the powder and the water in the mold to create a topical solution. The method may yet further include placing an applicator stick into the topical solution, the applicator stick configured to be used as an applicator for manipulating the topical solution after the topical solution has frozen thereon. The method may still yet further include placing the mold into a cooler or freezer, and then removing the mold from the cooler or freezer after the topical solution has at least partially frozen. The method may also include using the stick to remove the frozen topical solution from the mold and apply the frozen topical solution to a face of a user to deposit a mask of the topical solution. The method may further include allowing the mask to remain on the face, and then optionally removing the mask from the face.

According to yet another example embodiment, a kit is provided. The kit may include a mold with a cavity configured to hold a predetermined capacity of a liquid or semi-solid. The kit may further include an applicator configured to partially fit inside the mold wherein, when the mold is filled with the liquid or semi-solid to the predetermined capacity, a first portion of the applicator is submerged within the liquid or semi-solid and a second portion of the applicator is exposed. The mold may be configured to retain structural integrity at a range of temperatures at least between a freezing point of water and a customary room temperature and the first portion of the applicator may be configured to attach to a frozen portion of the liquid or semi-solid as it freezes. The second portion of the applicator may be configured to be manipulated by hand to apply the frozen portion of the liquid or semi-solid to skin of a subject.

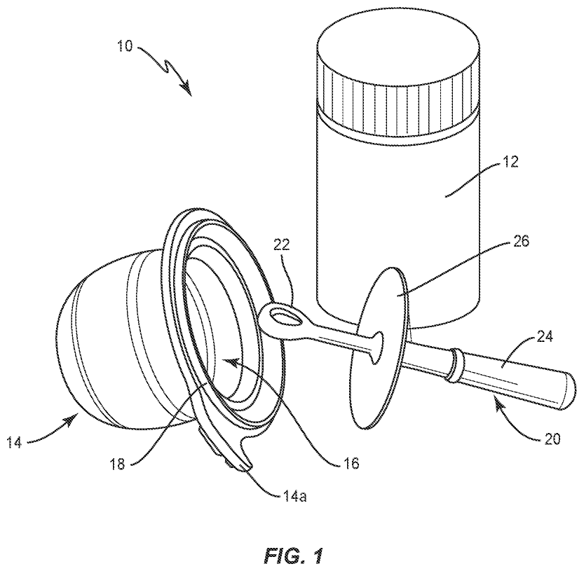

FIG. 1 depicts a diagram of components of a facial treatment system or “kit” 10, according to an example embodiment. The kit 10 includes an emollient product 12 such as a powder or a topical solution or other treatment, especially during cooling of the topical solution. There is a mold 14 into which an emollient fluid (“tropical solution”) is disposed. In some embodiments, the mold 14 comprises a concave portion or cavity 16 to receive the topical solution, with a rim 18 around the cavity. The mold 14 may also be formed with a tab 14a sized to be grasped by the user's fingers, to facilitate handling. There is an applicator 20 having a first portion 22, a second portion 24 and a flange 26 between the first portion and the second portion. The applicator 20 is configured so that the flange 26 can engage the rim 18 with the first portion 22 disposed within the cavity 16, so that the first portion 22 can be disposed in an emollient fluid and the flange 26 can seal the cavity to prevent leakage while the emollient fluid cools. Optionally, the form of the concave portion 16 can have a lower basin that is substantially hemispherical. Accordingly, the resulting frozen topical solution can have a rounded shape suitable for application to the skin with reduced risk for cutting, tearing, scraping, or otherwise damaging the skin during application. Other cavity shapes can be used to create specialized pops. For example, a smaller or slender cavity can be used create a pop specialized for more targeted or pin-point application.

In some embodiments, the cavity can be sized for, or have a capacity corresponding to, a single facial treatment, for example, a capacity between one to six ounces.

In some embodiments, the mold can made be from one or more flexible materials like silicone. Silicone can be flexible yet durable, so that the mold can washed by hand or machine (e.g., a home dishwasher) and reused many times. Moreover, a flexibility of the mold can aid in removal of the frozen topical solution from the mold, for example, by allowing a user to break a seal or contact between walls of the cavity and the frozen topical solution.

In another embodiments, the mold can comprise a more rigid material, such as hard plastic or metal, or be a combination flexible and rigid materials. For embodiments where the topical solution will be frozen, the mold can be of a material and quality capable of maintaining structural integrity at the range of temperatures the mold will experience, for example, between a freezing point of water and room temperature. In some embodiments, the mold can be microwavable or microwave-safe as well.

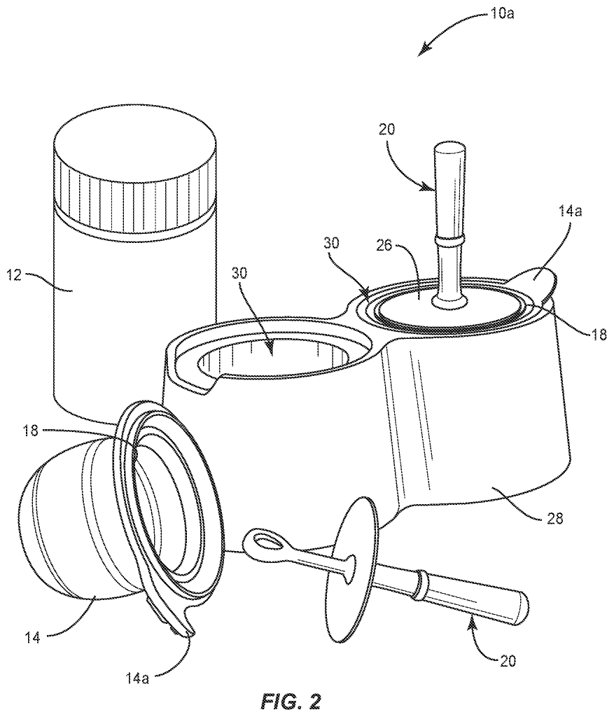

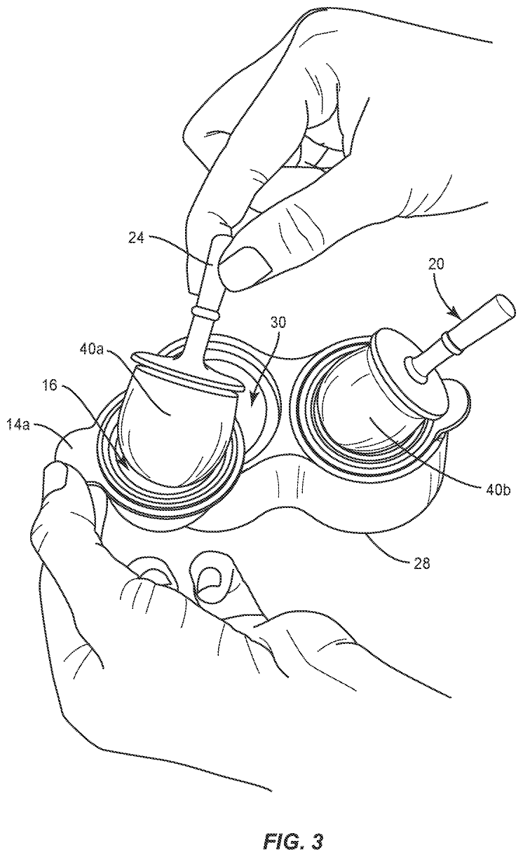

In alternative embodiments, a kit includes a plurality of molds 14 and associated applicators 20 or, as shown in FIG. 2, a kit may include a tray 28 having multiple wells 30a, 30b, each configured to receive and support a mold 14 for collective cooling and storage. Thus, two or more pops can be prepared simultaneously, optionally for use as part of a single treatment. Moreover, in some embodiments, the two or more pops can even have different formulations. The differently formulated pops can be used to treat different areas, or applied in overlapping patterns or in predetermined sequence to provide certain therapeutic or cosmetic benefits. FIG. 3 shows a user removing a pop 40a from a first mold 14, which is facilitated by grasping the tab 14a. FIG. 3 also shows a pop 40b partially removed from a second mold 14 in the tray 114.

The facial treatment system can optionally include a mixing unit for creating a topical solution to be applied to a subject. In some embodiments, a user may mix or otherwise create a topical solution by combining multiple ingredients to achieve a certain therapy profile. For example, the user may choose to include ingredients providing a cooling sensation, like mint, with an antioxidant ingredient to repair damaged skin. In some embodiments, a combination of ingredients may impart unique effects not present in, or present to reduced effect, in the individual ingredients themselves.

In some embodiments, ingredients in powdered form may be dissolved into or otherwise combined with water or another liquid. Multiple liquids can also be combined, and the mixture can be a solution or suspension. Moreover, an amount, type, and ratio of liquids and or solids can be chosen to give the mixture a desired freezing point and texture when applied. For home use, a target freezing point with a range of temperatures achievable by consumer refrigerators can be convenient for the user.

In some embodiments, the topical solution may be provided, packaged, or sold to a user as a premixed solution. In an example embodiment, the premixed solution can be a fixed amount corresponding to one or more treatments or doses. In another embodiment, the user may still have to measure out a predetermined amount of the premixed solution, for example, by filling a mold to the top of a cavity or predetermined mark on the mold. A premixed solution can also still require stirring or agitation prior to freezing to improve results.

In another embodiment, the ingredients for the emollient product can be individually wrapped or otherwise distinctly packaged to still require mixture or combination by the user, but can come can prepackaged in the appropriate amounts or ratios for use without measurement or waste. In some embodiments, the topical solution can be mixed while in the mold. The topical solution can even be sold or packaged inside the mold, with the user just needing to perform one or more of mixing and cooling to prepare a pop. Accordingly, in one embodiment, a premixed and packaged topical solution can be purchased and placed in a refrigerator substantially as packaged to prepare a pop.

In various embodiments, the topical solution can be, comprise, or function as one or more of a serum, face scrub, or face wash, wax, balm, and butter, lotion, or gel. The individual ingredients or a combination can be formulated or mixed to treat a pore size of the face, depuff the face, reduce inflammation, reduce swelling, treat acne; to cleanse, moisturize, exfoliate the skin, or reduce wrinkles; to induce a cooling, warming, or tingling sensation; to provide an anesthetic or antimicrobial effect; or to impart certain nutrients.

In some embodiments, the effects or effectiveness of certain ingredients can be moderated by a temperature of the topical solution. For example, an ingredient could be inert at a first temperature, e.g., when frozen, but activate as it warms to approach room temperature or body temperature. The topical solution can then be applied a temperature corresponding to the desired effect.

In some embodiments, an ingredient can be included in the topical solution to aid in removal of the topical solution from the mold. For example, an oil-based ingredient or lubricating agent could be added to help keep frozen topical solution from sticking to the mold.

The facial treatment system can also include an application unit. In some embodiments, the application unit, or applicator, can be used to apply the frozen topical solution to a subject, as shown in FIG. 4. In some embodiments, the applicator is an elongated member that is attached to the topical solution through the freezing process. For example, one end of the applicator can be embedded in the frozen topical solution, and another end exposed, for example, for manual manipulation by a user (e.g., as an applicator). The applicator and frozen topical solution together can form a pop.

In some embodiments, the elongated member can resemble a plain stick. In other embodiments, the applicator can include certain features to aid usability. For example, the applicator of the applicator may have a certain texture or expand in diameter further away from the frozen solution to improve grip. Other physical features can be incorporated to improve grip. In some embodiments, a middle section of the applicator may have a flange extending outward from the applicator that serves as a guard. The guard can keep the topical solution from dripping onto the hand of a user or the floor as the topical solution thaws or is deposited on a subject during treatment. In some embodiments, the flange can serve as a lid to the mold or cavity. The lid can cover or seal the mold so that the topical solution is covered during cooling and storage. The lid can also interface with the mold or cavity in a way that maintains an optimal position of the applicator inside the mold during freezing. Accordingly, at least a portion, and optionally an entirety, of one end of the applicator embedded in the topical solution while the topical solution is cooled to form a pop thereon. Preferably, the end of the applicator is positioned centrally in the mold so that the pop is formed symmetrically around the applicator.

In some embodiments, the embedded end of the applicator has a physical feature such as a retainer flange or tapered first portion to give the applicator an improved attachment to or grip on the frozen topical solution. FIG. 2 shows an applicator end with an expanding ring on one end. In some embodiments, the applicator can be used to stir or mix the topical solution. Accordingly, the end of the applicator may be configured to have a spoon or paddle-like end to facilitate mixing of the topical solution.

In an example embodiment, the applicator is at least partially formed of a rigid material such as a hard plastic or metal. The rigidity can give the pop enough stiffness such that the user can apply varying pressure and control when applying the treatment. In another example embodiment, the applicator is formed of silicone, or a rigid material covered by silicone or another flexible or soft material. Accordingly, there is reduced risk of the applicator damaging the skin of the subject, for example, should the applicator come in contact with the subject's skin.

In some embodiments, frozen topical solution can be removed from a mold or cavity using the applicator of the applicator. In another embodiment, the mold can have a tab or portion extending from an outside wall of the mold or cavity. A user can use the applicator of the applicator in conjunction with the tab of the mold to help separate the frozen topical solution and mold as shown in FIG. 3.

In some embodiments, the facial treatment system can also include a cooling device. In an example embodiment, the cooling device is a freezer or refrigeration unit capable of lowering an ambient temperature to a predetermined range or setting. For water-based topical solutions, this setting could be at or below a freezing point of water. In an example embodiment, the cooling device is a consumer refrigerator. In such embodiments, a mold or tray holding the topical solution could be placed inside the freezer or other appropriate section of the refrigerator. In another embodiment, the cooling unit can be a commercial unit configured to achieve lower temperatures than typical consumer refrigerators. In certain embodiments, deep freezing or flash freezing can be used to cool the topical solution. These techniques may improve the texture of the pop or reduce cooling time. In another embodiment, a custom cooling unit for holding a tray or mold can be used or even be incorporated into the tray or mold itself. For example, a tray incorporating a Peltier-based cooler could be used to quickly chill the topical solution to the desired temperature range.

In an example facial treatment method, a topical solution is placed into container to be cooled. As described further herein, the topical solution can be packaged, sold, obtained, or purchased pre-mixed, or created by a user by crushing, blending, or otherwise mixing their desired ingredients as part of pop preparation.

The topical solution in the container is cooled to a temperature corresponding to the desired cosmetic or therapeutic effect. In some embodiments, the topical solution is cooled to a point where it at least partially freezes. In an example embodiment, the frozen topical solution is substantially one whole solid. In another embodiment, the frozen topical solution can be more slush-like. In yet another embodiment, the topical solution does not undergo a phase change but is still cooled to a temperature below either a body (or skin) temperature of a subject, or below a customary room temperature or a room temperature of the treatment environment.

Once the topical solution is sufficiently cooled, (e.g., frozen), the cooled or frozen topical solution can be removed from the container and applied to the skin. In some embodiments, the frozen topical solution can be moved across the face while in contact with the face. As the frozen topical solution warms responsive to body heat transmitted through the skin of the face, the topical solution can thaw and deposit on the face. In another embodiment, friction is sufficient to cause deposition of the topical solution without thawing.

In some embodiments, a substantially constant pressure is applied by the user. In other embodiments, variable pressure may be used to “paint” the face. A speed or movement of the frozen topical solution across the face can be moderated to achieve a desired therapeutic effect. For example, lowering a temperature of the skin and underlying tissue to achieve vasoconstriction may require keeping the frozen topical solution in substantially the same treatment area longer. However, keeping the frozen topical solution in contact with an area of the skin for too long may cause undesirable trauma. Moreover, certain areas of the skin may more sensitive or in worse condition than others. A skilled user can account for local skin and tissue variations and use different techniques to target unique problem areas. For example, an area around the eyes may be targeted to treat the appearance of circles or bags under the eyes. However, the skin may be thinner around the eyes and getting topical solution into the eyes themselves may cause unwanted effects for the subject. Also a user may move the applicator in circular or other motions frequently to constantly to target desired treatment areas without letting the pop remain in one place on the skin for too long.

Accordingly, in some embodiments, an applicator is partially submerged or otherwise placed into the topical solution before freezing so that the non-submerged portion of the applicator can be used an applicator to manipulate the frozen topical solution on the subject's face. The applicator can give the user greater control cover the application. In some embodiments, with or without an applicator, the facial treatment may include massage of the topical solution into the skin, for example to promote lymph node drainage. In some embodiments, the frozen topical solution is molded into a tool itself for applying to skin without an applicator.

In some embodiments, the frozen topical solution or the skin of the subject can be treated before application to reduce the risk of skin trauma from the treatment or make application easier or more effective. For example, the frozen topical solution can be allowed to sit outside the cooling unit or. to warm for a predetermined amount of time or until it reaches a predetermined temperature, so that the frozen topical solution can thaw slightly before application. In another embodiment, water or a lubricant can be applied to the subject's face so that the pop does not stick in place or catch on the skin during application. In some embodiments, the pop may be rinsed with water, microwaved or otherwise warm to speed thawing of the frozen topical solution.

Once the treatment areas on the face are covered or otherwise treated, the deposited topical solution may form a “mask” on the face. In some embodiments, the mask is promptly removed. In other embodiments, the mask is left on removed after a predetermined amount of time, for example, five minutes. The interlude can let a mask dry, harden, or solidify so that it can be removed more easily, for example, by peeling, or can give the topical solution time to apply its desired effect, for example, chemical exfoliation. In various embodiments, the mask is removed by one or more of washing the skin, toweling the skin, wiping the skin, or rubbing the skin. However, in some embodiments, the mask may be left on indefinitely or to be washed off or removed according to a subject's natural cleaning routine.

### Example

An emollient powder was prepared by combining the following commercially obtained plant powders:

Musa spp (banana) powder fruit extract, (Banana juice powder) 97 g
Carica Papaya (papaya) powder fruit extract 18 g
Kasturi Manjal (wild turmeric) powder, 2.5 g
Aloe Barbadensis organic (aloe vera) powder, 2.5 g

To prepare an emollient fluid from the emollient powder, 10 milliliters (ml) of the emollient powder was deposited into a silicon mold, and 10 ml water is added to the mold. The water and emollient powder were mixed together at room temperature (about 70° F. (about 21° C.)) to form an emollient fluid of homogeneous consistency. The first portion of an applicator as described herein was inserted into the mold and the mold sealed by the flange on the applicator with the second portion of the applicator extending out from the mold to serve as a handle. The mold containing the emollient fluid was placed in the freezer of a conventional home refrigerator/freezer at a temperature about 0° F. (about −16° C.)). After about four hours in the freezer, the mold was examined and a pop was found to be formed on the applicator.

The mold containing the pop was removed from the freezer the applicator was used to remove the pop from the mold. The user then employed the handle to contact the pop with the skin, leaving on the skin a layer of emollient fluid which melted off the pop. After a time, the user removed the layer from the skin.

The temperature of the freezer is not critical as long as it is cold enough to allow the pop to form. A user may remove a layer of emollient fluid from the skin after any desired period of time, but from about 1 to about 15 minutes is expected to be typical for satisfactory results.

In some embodiments, the topical solution may give a visual or other indication that is dry or ready to be removed, for example by changing color.

In some embodiments, application of the topical solution is followed by another skin treatment or procedure. For example, a facial treatment could be used to reduce the apparent size of pores in the face. Makeup could then promptly be applied to the subject while the effects of the facial treatment endure.

While certain embodiments of the disclosed invention have been described in connection with what is presently considered to be the most practical and various embodiments, it is to be understood that the disclosed invention is not to be limited to the disclosed embodiments, but on the contrary, is intended to cover various modifications and equivalent arrangements included within the scope of the appended claims. Although specific terms are employed herein, they are used in a generic and descriptive sense only and not for purposes of limitation.

This written description uses examples to disclose certain embodiments of the disclosed invention, including the best mode, and also to enable any person skilled in the art to practice certain embodiments of the disclosed invention, including making and using any devices or systems and performing any incorporated methods. The patentable scope of certain embodiments of the disclosed invention is defined in the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

As shown throughout the drawings, like reference numerals designate like or corresponding parts. While illustrative embodiments of the present disclosure have been described and illustrated above, it should be understood that these are exemplary of the disclosure and are not to be considered as limiting. Additions, deletions, substitutions, and other modifications can be made without departing from the spirit or scope of the present disclosure. Accordingly, the present disclosure is not to be considered as limited by the foregoing description.

We claim:

1. A method for applying an emollient fluid to the skin, comprising:
cooling the emollient fluid to a temperature below room temperature; and
contacting the cooled emollient fluid to the skin.

2. The method of claim 1 wherein the emollient fluid comprises a volume of powdered plant product mixed into an approximately equal volume of an aqueous medium.

3. The method of claim 1 comprising cooling the emollient fluid to a solid emollient; and contacting the solid emollient to the skin to thaw a portion of the solid emollient and deposit a layer of the emollient fluid on the skin.

4. The method of claim 3 comprising providing an applicator having a first portion and a second portion and inserting the first portion of the applicator into the emollient fluid before cooling the emollient fluid, and cooling the emollient fluid while the second portion of the applicator extends out from the fluid, to form the solid emollient on the first portion of the applicator, and grasping the second portion of the applicator to contact the solid emollient with the skin.

5. The method of claim 2 comprising forming an emollient fluid by combining the powdered plant product with the aqueous medium.

6. The method of claim 5 comprising disposing the emollient fluid in the concave portion of a mold;
inserting the first portion of an applicator into the emollient fluid in the concave portion of the mold with a second portion of the applicator extending out from the fluid;
cooling the emollient fluid in the mold to a solid emollient on the first portion of the applicator;
removing the solid emollient and the applicator from the mold; and
using the second portion of the applicator to contact solid emollient with the skin.

7. The method of claim 6 wherein the powdered plant product comprises powdered banana fruit extract; powdered papaya fruit extract; powdered turmeric, and powdered aloe vera.

8. The method of claim 7 wherein the powdered plant product comprises about 80% powdered banana fruit extract, about 15% powdered papaya fruit extract, about 2.5% powdered tumeric and about 2.5% powdered aloe vera, by weight of the powdered plant product.

9. A skin treatment kit comprising:
a mold having a concave portion;
an applicator having a first portion and a second portion; and
an emollient product.

10. The skin treatment kit of claim 9 wherein the emollient product comprises a powdered plant product.

11. The skin treatment kit of claim 10 wherein the powdered plant product comprises powdered banana fruit extract; powdered papaya fruit extract; powdered turmeric, and powdered aloe vera.

11. The skin treatment kit of claim 10 wherein the powdered plant product comprises about 80% powdered banana fruit extract, about 15% powdered papaya fruit extract, about 2.5% powdered tumeric and about 2.5% powdered aloe vera, by weight of the powdered plant product.

12. The skin treatment kit of claim 10 wherein the powdered plant product comprises one or more powdered dried plant or powdered dried plant extracts selected from the group comprising pineapple, pumpkin, rice, banana, oats, cucumber, strawberry, lemon peel, watermelon, guava, mango avocado, tomato, blueberries, pomegranate, green tea (matcha), spirulina, dragon fruit, and beet.

13. The skin treatment kit of claim 9 wherein the emollient product comprises an emollient fluid comprising powdered plant product mixed into an approximately equal volume of an aqueous medium.

14. The skin treatment kit of claim 13 wherein the powdered plant product comprises powdered banana fruit extract; powdered papaya fruit extract; powdered turmeric, and powdered aloe vera.

15. The skin treatment kit of claim 14 wherein the powdered plant product comprises about 80% powdered banana fruit extract, about 15% powdered papaya fruit extract, about 2.5% powdered tumeric and about 2.5% powdered aloe vera, by weight of the powdered plant product.

16. The skin treatment kit of claim 9 wherein the mold has a concave portion and a rim surrounding the concave portion, and the applicator comprises a closure flange between the first portion and the second portion, and the closure flange is configured to removably engage the rim while the first portion of the applicator is disposed in the concave portion of the mold.

17. The skin treatment kit of claim 10 wherein the powdered plant product comprises one or more powdered dried plant or powdered dried plant extracts selected from the group comprising pineapple, pumpkin, rice, banana, oats, cucumber, strawberry, lemon peel, watermelon, guava, mango avocado, tomato, blueberries, pomegranate, green tea (matcha), spirulina, dragon fruit, and beet.

18. The skin treatment kit of claim 13 wherein the powdered plant product comprises one or more powdered dried plant or powdered dried plant extracts selected from the group comprising pineapple, pumpkin, rice, banana, oats, cucumber, strawberry, lemon peel, watermelon, guava, mango avocado, tomato, blueberries, pomegranate, green tea (matcha), spirulina, dragon fruit, and beet.
